(19) United States
(12) Patent Application Publication
(10) Pub. No.: US 2019/0125804 A1
(43) Pub. Date: May 2, 2019

(54) ANTI-CANCER USE OF GENETICALLY MODIFIED HUMAN UMBILICAL CORD PERIVASCULAR CELLS (HUCPVC)

(71) Applicant: Tissue Regeneration Therapeutics Inc., Toronto (CA)

(72) Inventors: Catalina Estrada Vallejo, Toronto (CA); John E. Davies, Toronto (CA)

(21) Appl. No.: 16/309,724

(22) PCT Filed: Jun. 14, 2017
(86) PCT No.: PCT/CA2017/000148

§ 371 Date: Dec. 13, 2018

Related U.S. Application Data
(60) Provisional application No. 62/350,460, filed on Jun. 15, 2016.

(51) Int. Cl. A61K 35/51; A61K 39/395; C07K 16/22; C07K 16/32; A61K 38/20; A61K 38/21; A61K 38/17; C07K 16/44; C12N 15/113

(52) U.S. Cl. CPC A61K 35/51; C12N 2320/32; C07K 16/22; C07K 16/32; A61K 38/2013; A61K 38/212; A61K 38/177; A61K 38/1709; A61K 39/39558; C07K 16/44; C12N 15/1135; A61K 38/2066; C07K 2317/24; C12N 2310/14; A61K 39/3955

(57) ABSTRACT

Herein described is a method for treating cancer in a subject by administering a human umbilical cord perivascular cell (HUCPVC) that has been genetically modified to increase the expression of an oligonucleotide or a polypeptide such as an anti-cancer antibody.

### FIELD OF THE INVENTION

The invention provides methods of preventing or treating cancer by administering genetically modified human umbilical cord perivascular cells. Also provided are genetically modified human umbilical cord perivascular cells useful in such a method, and pharmaceutical compositions comprising them.

### BACKGROUND OF THE INVENTION

For use in cancer treatment, recombinant proteins, like antibodies, are produced by a costly process involving large scale cGMP manufacturing and eukaryotic/prokaryotic fermentation systems, followed by downstream purification and formulating the bulk active ingredient. The large scale use of recombinant proteins via conventional and often repeated injection or infusion can be impractical. Particularly in the treatment of cancer, toxicity that results from multiple and even single administrations creates severe difficulties for patients, and makes patient compliance difficult to manage.

### SUMMARY OF THE INVENTION

The invention provides a method and means that can simplify protein drug therapy in cancer treatment and can reduce costs by eliminating the need for their large scale production, by administering a human umbilical cord perivascular cell (HUCPVC) that has been genetically modified to increase the expression of an anti-cancer oligonucleotide or polypeptide. Also provided, in another aspect of the invention, are genetically modified HUCPVCs useful in such a method, and pharmaceutical compositions and articles of manufacture that comprise such genetically modified HUCPVCs.

The subject can be a vertebrate, such as a mammal (e.g., a human). The HUCPVC can be allogeneic or xenogeneic relative to the subject to which it is administered.

In an embodiment of the invention, genetically modified HUCPVCs are administered to a subject to treat cancer. Cancers that can be treated are those that respond to a protein produced by expression from a transgene incorporated within a HUCPVC host. As a human cell itself, and one that is like a mesenchymal stem cell in its nature, the genetically modified HUCPVC is useful to produce a wide and relevant variety of anti-cancer proteins.

In another embodiment of the invention, genetically modified HUCPVCs are administered to a subject to treat cancer, such as by controlling cancer cell activity.

In another embodiment of the invention, HUCPVCs are genetically modified to produce an oligonucleotide having anti-cancer activity. In another embodiment of the invention, HUCPVCs are genetically modified to produce and secrete a polypeptide having anti-cancer activity.

In a particular embodiment, HUCPVCs are genetically modified to produce an anti-cancer antibody or fragment thereof. The antibody or antibody fragment can be recombinant, humanized, or monoclonal. The antibody or antibody fragment can be a single chain antibody (scFv), Fab, Fab′2, scFv, SMIP, diabody, nanobody, aptamer, or domain antibody.

In another particular embodiment of the invention, HUCPVCs are genetically modified to produce a polypeptide that inhibits interaction between a growth factor and its receptor. The inhibitor can be a chimeric protein or a fusion protein such as an Fc fusion protein. The inhibitor can be a fragment or variant of a polypeptide member of a binding pair involved in cancer progression. For instance, the inhibitor can target the epidermal growth factor receptor (EGFR)/ligand interaction, and can be a fragment of epidermal growth factor (EGF) or heparin binding epidermal growth factor (HB-EGF) that binds the EGF receptor, or a fragment of the extracellular region of the EGFR. Similarly, the cells can be modified to produce inhibitors, e.g., antibodies, against growth factor interactions based on transforming growth factor-beta (TGF-β), platelet derived growth factor (PGDF), insulin-like growth factor (IGF), glucagon-like peptide 2 (GLP-2), vascular endothelial growth factor (VEGF), and keratinocyte growth factor (KGF), for example.

In another embodiment, the polypeptide can be an anti-cancer antibody that binds to a growth factor or growth factor receptor, such that signaling across that axis is inhibited. The growth factor receptor can be the receptor protein expressed from the human epidermal growth factor receptor 2, (her-2) gene. In one embodiment, the anti-cancer antibody is trastuzumab and/or pertuzumab. In a different embodiment, the anti-cancer polypeptide produced by the modified HUCPVCs is an antibody that binds to the epidermal growth factor receptor and is selected from cetuximab, panitumumab, matuzumab, nimotuzumab, zalutumab and necitumumab. In a specific embodiment, the anti-cancer agent is the her-2 antibody known as trastuzumab, and the HUCPVC host is transfected to express the two polypeptide chains that together form this antibody.

In a further particular embodiment, HUCPVCs are genetically modified to produce a polypeptide that inhibits an immune checkpoint. In embodiments, the inhibitor is a protein that inhibits signaling via the PD-1 receptor. In an embodiment, the protein is an antibody. In an embodiment, the protein is a PD-1 receptor antibody. In other embodiments, the antibody binds selectively to one of CTLA-4, PD-1, PD-L1, PD-L2, CD20, CD40, CD47, SIRPa, toll-like receptors TLR3, TLR7, TLR8, CD200, VCP, PLIF, LSF-1, Nip, uromodulin, CD40L (CD154), FasL, CD27L, CD30L, CD47, SIRPα, CD28, CD25, B7.1, B7.2, and OX40L.

The anti-cancer polypeptide can also be an anti-cancer interferon such as interferon-alpha (IFN-α), or an IFN-γ, or an interleukin such as interleukin-2, or a polypeptide of the TRAIL family. The anti-cancer polypeptide can also be an inhibitor of soluble CD4, cystic fibrosis transmembrane conductance receptor (CFTR), or an Fc receptor; or an inhibitor of an immunomodulating protein listed above.

In yet other embodiments, the same population of HUCPVCs can be genetically modified to express two or more polypeptides that are separately active as anti-cancer agents or that together combine to provide anti-cancer activities, such as antibodies. That is, the HUCPVC can be modified genetically to produce two or more polypeptides, or the subject can be treated by administration of two or more different HUCPVC each producing a different anti-cancer polypeptide. For example, a subject can be treated with cells that provide a combination of trastuzumab and pertuzumab, either in the same cell or in two different cells. Thus, the anti-cancer polypeptides can be introduced to the subject by administration of different HUCPVC populations.

In yet other embodiments of the invention, the HUCPVCs are genetically modified to express an oligonucleotide, e.g., an RNA interference (RNAi) molecule capable of inhibiting oncogene expression. The RNAi molecule can be a small inhibitory RNA (siRNA) or short hairpin RNA (shRNA) molecule. The oligonucleotide can be endogenous or non-endogenous to the HUCPVC. In other embodiments, the HUCPVCs can be genetically modified to express two or more oligonucleotides.

In another embodiment of the invention, the subject presents with a cancer before receiving treatment with a genetically modified HUCPVC of the invention. The subject can be administered a single dose of HUCPVCs or multiple doses of HUCPVCs. The HUCPVCs can be administered as an anti-cancer immunogen/vaccine to protect a subject in need thereof. The HUCPVC can be administered to a subject intravenously, intramuscularly, orally, by inhalation, parenterally, intraperitoneally, intraarterially, transdermally, sublingually, nasally, buccally, liposomally, adiposally, opthalmically, intraocularly, subcutaneously, intrathecally, topically, or locally. The subject can be administered between 10¹ and 10³ HUCPVCs per dose, or between 10³ and 10⁸ HUCPVCs per dose.

In other embodiments of the invention, genetically modified HUCPVCs administered to a subject persist for greater than one week, one month, two months or six two months. The HUCPVCs can evade immune recognition in the subject.

In yet other embodiments of the invention, genetically modified HUCPVCs are administered in combination with at least one mesenchymal stem cell (MSC) that is not a HUCPVC. The MSC can be genetically modified to increase the expression of an oligonucleotide or a polypeptide in the MSC relative to a MSC that has not been genetically modified. The MSC can be isolated from bone marrow, adipose tissue, umbilical cord blood, embryonic yolk sac, placenta, skin, or blood. The MSCs can be genetically modified to express an oligonucleotide or polypeptide that is endogenous or non-endogenous to the HUCPVC.

In another embodiment of the invention, genetically modified HUCPVCs can be administered to a subject in combination with one or more therapeutic agents that enhance or prolong the therapeutic effect of HUCPVC treatment. The therapeutic agent can be any of the agents identified above, e.g., an anti-cancer protein such as an antibody or antibody fragment that blocks interactions involving growth factor receptors such as those expressed from such genes as HER-1 (EGFR), HER-2, HER-3 and HER-4, and receptors for PDGF, VEGF, etc., as well as proteins that serve as immune checkpoint inhibitors, and proteins that are inhibitors of growth factor receptors and their ligands such as TNFα, as well as TGFβ, some interleukins such as IL-2 and IL-10, as well as interferons, etc. as well as standard drug therapies used in cancer treatments.

In yet another embodiment of the invention, genetically modified HUCPVCs are administered with a pharmaceutically acceptable carrier or excipient.

In another embodiment of the invention, genetically modified HUCPVCs are provided in a kit for administration to a subject in need of treatment of or protection from biological or chemical agents.

In general, the present invention provides for the use of HUCPVCs that are genetically modified for the preparation of a medicament for preventing or treating diseases or disorders caused by cancer in a subject. The genetically modified HUCPVCs per se are also provided by the present invention, defined as novel HUCPVCs that are genetically modified to express anti-cancer agents, and particular those agents that are not useful as countermeasures.

### Definitions

The term “antibody” as used interchangeably herein, includes whole antibodies or immunoglobulins and any antigen-binding fragment or single chains thereof. Antibodies, as used herein, can be mammalian (e.g., human or mouse), humanized, chimeric, recombinant, synthetically produced, or naturally isolated. Antibodies of the present invention include all known forms of antibodies and other protein scaffolds with antibody-like properties. For example, the antibody can be a human antibody, a humanized antibody, a bispecific antibody, a chimeric antibody, or a protein scaffold with antibody-like properties, such as fibronectin or ankyrin repeats. The antibody also can be a Fab, Fab′2, scFv, SMIP, diabody, nanobody, aptamers, or a domain antibody. The antibody can have any of the following isotypes: IgG (e.g., IgG1, IgG2, IgG3, and IgG4), IgM, IgA (e.g., IgA1, IgA2, and IgAsec), IgD, or IgE. The antibody desirably is an IgG1 or IgG4 antibody.

The term “antibody fragment”, as used herein, refers to one or more fragments of an antibody that retain the ability to specifically bind to an antigen. The antigen-binding function of an antibody can be performed by fragments of a full-length antibody. Examples of binding fragments encompassed within the term “antigen-binding portion” of an antibody include but are not limited to: (i) a Fab fragment, a monovalent fragment consisting of the V_{L}, V_{H}, C_{L}, and C_{H}1 domains; (ii) a F(ab′)₂ fragment, a bivalent fragment comprising two Fab fragments linked by a disulfide bridge at the hinge region; (iii) a Fd fragment consisting of the V_{H} and C_{H}1 domains; (iv) a Fv fragment consisting of the V_{L} and V_{H} domains of a single arm of an antibody, (v) a dAb including V_{H} and V_{L} domains; (vi) a dAb fragment (Ward et al., Nature 341:544-546 (1989)), which consists of a V_{H} domain; (vii) a dAb which consists of a V_{H} or a V_{L} domain; (viii) an isolated complementarity determining region (CDR); and (ix) a combination of two or more isolated CDRs which may optionally be joined by a synthetic linker. Furthermore, although the two domains of the Fv fragment, V_{L} and V_{H}, are coded for by separate genes, they can be joined, using recombinant methods, by a synthetic linker that enables them to be made as a single protein chain in which the V_{L} and V_{H} regions pair to form monovalent molecules (known as single chain Fv (scFv); see e.g., Bird et al., Science 242:423-426 (1988) and Huston et al., Proc. Natl. Acad. Sci. USA 85:5879-5883 (1988)).

The term “human antibody,” as used herein, is intended to include antibodies, or fragments thereof, having variable regions in which both the framework and CDR regions are derived from human germline immunoglobulin sequences as described, for example, by Kabat et al., (Sequences of proteins of Immunological Interest, Fifth Edition, U.S. Department of Health and Human Services, NIH Publication No. 91-3242 (1991)). Furthermore, if the antibody contains a constant region, the constant region also is derived from human germline immunoglobulin sequences. The human antibodies may include amino acid residues not encoded by human germline immunoglobulin sequences (e.g., mutations introduced by random or site-specific mutagenesis in vitro or by somatic mutation in vivo). However, the term “human antibody”, as used herein, is not intended to include antibodies in which CDR sequences derived from the germline of another mammalian species, such as a mouse, have been grafted onto human framework sequences (i.e., a humanized antibody or antibody fragment).

The term “humanized antibody” refers to any antibody or antibody fragment that includes at least one immunoglobulin domain having a variable region that includes a variable framework region substantially derived from a human immunoglobulin or antibody and complementarity determining regions (e.g., at least one CDR) substantially derived from a non-human immunoglobulin or antibody.

The terms “effective amount” or “amount effective to” or “therapeutically effective amount” means an amount of genetically modified HUCPVCs sufficient to produce a desired result, for example, treating cancer.

By “treating” is meant the reduction (e.g., by at least 10%, 15%, 20%, 25%, 30%, 35%, 40%, 45%, 50%, 60%, 70%, 80%, 90%, 95%, 99%, or even 100%) in the progression or severity or frequency of one or more features or symptoms of cancer in a subject (e.g., a human), or the improvement in the rate of proliferation, tumour volume, tumour distribution or overall tumour burden experienced by the treated subject. “Anti-cancer” is intended to mean that the described entity has the effect of treating a cancer, and a cancer sufferer. Also, the ability to “control” cancer means that there is an anti-cancer effect useful to treat the cancer.

By “genetically modified HUCPVC” is meant a human umbilical cord perivascular cell that recombinantly expresses at least one polypeptide (e.g., an antibody) or oligonucleotide (e.g., an siRNA) that, when administered to a human, can treat cancer. This polypeptide or oligonucleotide will be recombinantly produced by the HUCPVC following transfer (e.g., transfection or transduction) of the genetic sequence for the polypeptide or oligonucleotide to the HUCPVC.

By “pharmaceutically acceptable carrier” is meant a carrier which is physiologically acceptable to the treated subject (e.g., a human) while retaining the therapeutic properties of the genetically modified HUCPVCs with which it is administered. One exemplary pharmaceutically acceptable carrier is physiological saline. Other physiologically acceptable carriers and their formulations are known to one skilled in the art and described, for example, in Remington's Pharmaceutical Sciences, (18^{th} edition), ed. A. Gennaro, 1990, Mack Publishing Company, Easton, Pa. incorporated herein by reference.

A “transgene” is simply a gene, such as a polynucleotide or oligonucleotide, that encodes a protein or oligonucleotide of interest, such as a polypeptide or oligopeptide, and that is heterologous with respect to its expression host, particularly HUCPVCs. Thus, the transgene is usually obtained from a source other than HUCPVCs. A transgene can be a gene that is native to HUCPVCs but still is inserted into a HUCPVC to establish a desired effect in the host, such as overexpression of a desired gene product.

An “immune checkpoint” is an interaction that is responsible for maintaining balance in the immune system and its reaction to a cancer cell. Certain immune checkpoints are exploited by some cancer cells. The present invention accordingly includes agents that inhibit the interaction that constitutes the checkpoint, to block or inhibit a cancer cell's reliance on that interaction for its survival.

### BRIEF REFERENCE TO THE DRAWINGS

These and other aspects of the present invention are now described in greater detail with reference to the accompanying drawings, in which:

FIG. 1 shows production of trastuzumab IgG1 from different HEK293 populations, on such population comprising light chain-encoding DNA and the other population comprising heavy chain encoding DNA. As shown, expression of the dimeric antibody is increasing with time. Two different transfection protocols were tested; and

FIG. 2 shows the production of bioactive TRAIL by genetically modified HUCPVCs using adenovirus-based transfection. Note that production of TRAIL was not detected in conditioned media of naïve HUCPVCs.

FIG. 3 shows production of interleukin-10 by HUCPVCs.

### DETAILED DESCRIPTION OF THE INVENTION

The invention provides genetically modified human umbilical cord perivascular cells (HUCPVCs), medically useful compositions comprising them, and the administration thereof to inhibit, reduce, prevent, or treat cancer. In addition, genetically modified HUCPVCs are administered therapeutically to a mammal having cancer for the treatment thereof.

A HUCPVC can be genetically modified to express a gene that encodes any polypeptide (e.g., a human polypeptide), that is useful for the treatment of cancer. The polypeptide can be an inhibitor of a process that supports cancer progression, such as a dimeric protein including an Fc fusion or antibody or antibody fragment that binds to a cancer causing agent or process. The polypeptide can also be a monomeric polypeptide, such as a growth factor inhibitor or an immunomodulatory agent. In embodiments, the polypeptide can itself be the anti-cancer agent, by supplementing a deficiency that supports cancer progression. It will be appreciated that a very large number and variety of polypeptides have anti-cancer activity, and any of these can be embodiments of the present invention. To this end, there are numerous databases that catalog anti-cancer agents that are polypeptide in nature, and these can be consulted to identify the polypeptides useful herein, as well as their protein and gene sequences and their other properties (infra).

In addition, a HUCPVC can be genetically modified to express an oligonucleotide (e.g., an RNAi molecule) that modulates (e.g., inhibits) a cellular process of the treated subject or the cancer therein. A HUCPVC can be genetically modified to express one or more therapeutic polypeptides or oligonucleotides for the prevention or treatment of cancer.

Genetically modified HUCPVCs can be co-administered with one or more diagnostic or therapeutic agents to enhance or prolong the prophylactic or therapeutic qualities of the HUCPVC treatment. HUCPVCs can also be combined as a mixture comprising HUCPVC populations that differ in terms of the polypeptide they produce as a result of genetic modification. HUCPVCs can be administered with one or more pharmaceutically acceptable carriers or excipients and can be formulated to be administered intravenously, intramuscularly, orally, by inhalation, parenterally, intraperitoneally, intraarterially, transdermally, sublingually, nasally, transbuecally, liposomally, adiposally, opthalmically, intraocularly, subcutaneously, intrathecally, topically, or locally. In a further aspect, the invention provides a kit, and an article of manufacture, with instructions, for the therapeutic anti-cancer treatment of a mammal with one or more genetically modified HUCPVC populations.

### Human Umbilical Cord Perivascular Cells (HUCPVCs)

Human umbilical cord perivascular cells (HUCPVCs) are a non-hematopoietic, mesenchymal, population of multipotent cells obtained from the perivascular region of the blood vessels within the Wharton's Jelly of human umbilical cords (see, e.g., Sarugaser et al., “Human umbilical cord perivascular (HUCPV) cells: A source of mesenchymal progenitors,” Stem Cells 23:220-229 (2005)). U.S. Patent Application Publication 2005/0148074 and International Patent Application Publication WO 2007/128115 describe methods for the isolation and in vitro culture of HUCPVCs, and are incorporated by reference herein. HUCPVCs are further characterized by relatively rapid proliferation, exhibiting a doubling time, in each of passages 2-7, of about 20 hours (serum dependent) when cultured under standard adherent conditions. Phenotypically, the HUCPVCs are characterized, at harvest, as Oct 4−, CD14−, CD19−, CD34−, CD44+, CD45−, CD49e+, CD90+, CD105(SH2)+, CD73(SH3)+, CD79b−, HLA-G−, CXCR4+, and c-kit+. In addition, HUCPVCs are positive for CK8, CK18, CK19, PD-L2, CD146 and 3G5 (a pericyte marker), at levels higher relative to cell populations extracted from Wharton's jelly sources other than the perivascular region. In simple terms the HUCPVCs are characterized phenotypically as 3G5+, CD45−, CD44+. They are extractable, for instances using enzymes such as collagenase, from the Wharton's jelly that surrounds the cord blood vessels, i.e., from perivascular tissue.

### Advantages of HUCPVCs

When used recombinantly to express a polypeptide or oligonucleotide (e.g., a human polypeptide or oligonucleotide), genetically modified HUCPVCs offer several advantages over other cell-based therapies. Because HUCPVCs exhibit low immunogenicity when administered to an allogeneic or xenogeneic host, they have an increased longevity within the host relative to other allogeneic or xenogeneic cells. HUCPVCs also have established gene expression modalities that result in therapeutically significant levels of a protein or oligonucleotide of interest (e.g., a recombinant polypeptide or oligonucleotide that the HUCPVC has been genetically modified to express). In addition, although HUCPVCs proliferate rapidly, they have a reduced risk of proliferative disorders relative to other cell-based gene therapy vehicles. Each of these advantageous properties of genetically modified HUCPVCs for the prophylaxis or treatment of a subject (e.g., a human) is discussed below.

The low immunogenicity of genetically modified HUCPVCs makes them ideal as vehicles for administration to vertebrate subjects, e.g., mammals, such as humans, and particularly to allogeneic or xenogeneic recipients. HUCPVCs have been shown to have low immunogenicity based on their ability to avoid detection by the host immune system (see, e.g., Sarugaser et al., (2005) and U.S. Patent Application Publication 2005/0148074). As such, HUCPVCs harvested from, e.g., a human (i.e., a donor) may be cultured in vitro and administered to another, un-related and HLA-mismatched, human (i.e., a host) without eliciting an allo-specific immune response in the host against the genetically modified HUCPVCs (see, e.g., Ennis et al., “In vitro immunologic properties of human umbilical cord perivascular cells” Cytotherapy 10(2):174-181 (2008)). Therefore, genetically modified HUCPVCs can be administered to heterologous human populations, or even to xenogeneic populations, without a loss of therapeutic efficacy due to activation of the host immune system. Furthermore, the ability to use HUCPVCs in virtually any vertebrate (e.g., a mammal, such as a human) allows for the large-scale preparation and storage (i.e., “stockpiling”) for subsequent use.

The low immunogenicity of HUCPVCs results in increased longevity of these cells in vivo in the treated host relative to other allogeneic or xenogeneic cells. Similar mesenchymal cells have been documented to persist in a human host for years when delivered allogeneically and thus, it can be expected that HUCPVCs will persist within a vertebrate (e.g., a mammalian, such as a human) host for at least weeks to months (e.g., 2 weeks, 4 weeks, 6 weeks, 2 months, 6 months or more) following injection. The longevity of HUCPVCs used to provide polypeptides or oligonucleotides for therapy or prophylaxis (e.g., by providing a viral polypeptide or oligonucleotide) offers benefits over other techniques of therapy. Whereas standard therapeutics require multiple administrations to confer a therapeutic effect in an individual, a therapeutically-effective amount of genetically modified HUCPVCs can instead be administered to an individual in a single dose. Alternatively, two or more doses of the genetically modified HUCPVCs can be administered to provide therapy.

Another advantageous property of HUCPVCs is that they can be genetically modified by a number of standard transfection and transduction techniques to allow for the recombinant expression of a therapeutic polypeptide or oligonucleotide. As described further herein, genetic transfer of a transgene can be achieved using viral vectors (e.g., adenoviruses and lentiviruses) and nucleic acid transfection (e.g., DNA plasmids in combination with liposomes, cationic vehicles, or electroporation).

Unlike many other mesenchymal stem cell populations that typically require the donation of bone marrow, HUCPVCs can be reliably collected from human umbilical cords that are normally discarded following birth. In industrialized nations, human umbilical cord blood products are now routinely collected and stored for possible future self or allo-transplantation. As such, the collection of HUCPVCs for expansion and genetic modification, according to the methods of the invention, are free of many of the logistical and ethical constraints associated with the collection of other mesenchymal stem cell populations.

Finally, HUCPVCs have a short population doubling time (see, e.g., Sarugaser et al., 2005) that allows for the rapid and properly scaled preparation of genetically modified HUCPVCs for administration to a mammal (e.g., a human) in need thereof. HUCPVCs substantially lack the enzyme telomerase, and therefore the risk of developing proliferative diseases is minimal as these cells cannot divide more than a prescribed number of divisions before apoptosis occurs. In animal experiments, HUCPVCs are not known to generate tumors, even when administered in numbers orders of magnitude larger than clinically applicable.

### Recombinant Polypeptide and Oligonucleotide Expression

In the present invention, HUCPVCs can be genetically modified to express one or more polypeptides (e.g., antibodies) or oligonucleotides (e.g., siRNA molecules) such that, when provided in a therapeutically-effective amount, the genetically modified HUCPVCs act themselves as drugs useful to inhibit, reduce, prevent or treat cancer. Anti-cancer oligonucleotides or polypeptides can also be expressed in HUCPVCs to improve host responses to the cancer. Polypeptides expressed in HUCPVCs can be secreted or displayed on the plasma membrane surface (e.g., a membrane-bound receptor or ligand). One or more oligonucleotides or polypeptides can be co-expressed in a single HUCPVC to allow for the treatment of one or more types or stages of cancer. Alternatively, the same patient can receive two or more HUCPVC populations, each one producing a recombinant polypeptide that is different but cooperative with the other in treating cancer. For instance, one HUCPVC population can produce trastuzumab and the other HUCPVC population can produce pertuzumab. Alternatively, the same HUCPVC transformant can produce both antibody drugs. There is a clinical preference for combining these particular drugs in the treatment of cancer, and this can be done efficiently when HUCPVC populations are used for this or any combination of two or more polypeptide drugs.

### Antibodies and Antibody Fragments

The invention further provides for the production of antibodies (e.g., humanized antibodies) or antibody fragments by genetically modified HUCPVCs that specifically bind anti-cancer targets. Exemplary anti-cancer antibodies include antibodies that bind to any surface marker or soluble product that is unique, in terms of its nature or its surface density, to the cancer cell relative to normal cells. This includes an enormous number and type of markers/antigens that can themselves be protein, glycoprotein, carbohydrate, or nucleic acid in composition, or mixtures thereof. In embodiments, the antibody is one that binds selectively to any known anti-cancer target.

Useful transgenes, for example, encode antibody to a growth factor receptor such as a receptor for any ligand within the transforming growth factor-beta (TGF-β) superfamily, platelet derived growth factor (PGDF), insulin-like growth factors (IGFs), epidermal growth factor (EGF), transforming growth factor (TGF-α), vascular endothelial growth factor (VEGF), hepatocyte growth factor (HGF), keratinocyte growth factor (KGF), glucagon-like peptide 2 (GLP-2) and the like. The growth factor ligands for each of these receptors can also be produced as anti-cancer polypeptides through HUCPVC genetic modification.

Other useful transgenes encode antibody to an immune checkpoint agent or immunomodulatory agent including CTLA-4, PD-1, PD-1 receptor, PD-L1, PD-L2, CD20, CD40, CD47, SIRPa, toll-like receptors TLR3, TLR7, TLR8, and CD200, as well as VCP, PLIF, LSF-1, Nip, uromodulin, CD40L (CD154), FasL, CD27L, CD30L, CD47, SIRPα, CD28, CD25, B7.1, B7.2, and OX40L, thus each of these and its binding partner can be produced by HUCPVCs for cancer treatment.

Antibodies and their active fragments that bind to these agents can be useful to neutralize them and the cancer processes in which they are involved. In addition, in any receptor/ligand interaction or any binding event at all, the anti-cancer polypeptides useful herein include soluble and non-functional (antagonistic) fragments of the proteins themselves. That is, useful anti-cancer polypeptides for HUCPVC production include fragments for instance of the epidermal growth factor receptor, the fragments being those able to bind the EGF ligand to inhibit its receptor interaction. Similarly, the protein can be an inactive fragment of EGF, the fragment having the ability to bind to the EGF receptor, thereby to disrupt ligand (EGF, TGFα, etc.)-mediated signaling across this axis.

Antibodies of this general type that can be produced by genetically modified HUCPVCs include EGFR antibodies cetuximab (Erbitux®), panitumumab (Vectibix®), matuzumab, nimotuzumab, necitumumab, zalutumab, ch806, 13.1, 13.1.2, 1024, 992, MM-151 and J2898A. (the antibody mixture known as MM-151 comprises 3 naked EGFR antibodies that include the antibodies described in Merrimack's U.S. Pat. No. 9,044,460, i.e., EGFR antibodies designated ca, cd and ch). The sequences of the CDRs for each antibody are provided in the US'460 patent, and are incorporated herein by reference. Public databases provide the sequences for each of the other species); and antibodies that bind to the expression product of the HER-2 gene associated with breast cancer such as trastuzumab and pertuzumab as well as HER-2 protein-binding fragments thereof;

Particular antibodies that target immune checkpoints and can usefully be produced by the genetically modified HUCPVCs include ipilimumab (CTLA-4), atezolizumab (PD-1), pembrolizumab or nivolumab (PD-1 receptor), rituximab (CD20), and ofatumumab (CD20). PD-L2, CD20 (rituximab, ofatumumab), and antibodies that target CD40, CD47, SIRPa, toll-like receptors TLR3, TLR7, TLR8, and CD200.

Antibodies against chemokines such as interleukin 2 that can be used in the present method include basiliximab and daclizumab.

Antibodies can also include those which bind to and inhibit a baculoviral IAP repeat-containing protein (BIRC) selected from BIRC1, BIRC2, BIRC3, BIRC4, BIRC5 (survivin), BIRC6, BIRC7 and BIRC8.

### Polypeptides as Anti-Cancer Agents

The invention further provides for the expression of polypeptides, rather than antibodies specifically, that are themselves directly effective to treat cancer when produced by HUCPVCs. This includes an enormous number and type of polypeptides, usually monomeric polypeptides but also Fc fusion versions thereof that can themselves be protein, glycoprotein, or nucleoprotein in composition. The Fc fusions are produced by expression from a gene that encodes an antibody Fc region fused to the polypeptide of interest, so that expression and then secretion from the host yields the Fe fusion protein as a dimeric product, comprising two molecules of the polypeptide of interest and an Fc region wherein the two Fc regions link the polypeptides through disulfide cross-linking.

In embodiments, the polypeptide is one that contributes an anti-cancer effect as an agonist, including such agents as:
An interferon that is either interferon-alpha (IFN-α) or interferon-gamma (IFN-γ) and their anti-cancer subspecies including IFN-α-2; and
A chemokine like an interleukin shown to have anti-cancer activity including interleukin-2; and
An agent including tumor necrosis factor (TNF), such as TNF-α; as well as agents that promote healthy cell proliferation such as granulocyte macrophage colony-stimulating factor (GM-CSF), granulocyte colony-stimulating factor (G-CSF), erythropoietin (EPO), and thrombopoietin (TPO); and
A TRAIL polypeptide family member, including related members of the TNF (tumour necrosis factor) family, as well as ligands of death receptors generally. TRAIL itself is “TNF-related apoptosis-inducing ligand” and is a protein functioning as a ligand that induces the process of cell death called apoptosis. TRAIL is a cytokine that is produced and secreted by most normal tissue cells. It causes apoptosis primarily in tumor cells by binding to certain death receptors. TRAIL also is named CD253 and TNFSF10 (tumor necrosis factor (ligand) superfamily, member 10); and
A decorin polypeptide family member, which embraces the small leucine-rich proteoglycans (SLRP). Decorin itself interacts with fibronectin, thrombospondin, the complement component C1q, epidermal growth factor receptor (EGFR) and transforming growth factor-beta (TGF-beta) and was shown to either enhance or inhibit the activity of TGF-β1. It inhibits angiogenesis by interaction with VEGFR2 (vascular endothelial growth factor receptor). Other angiogenic growth factors that decorin inhibits are angiopoietin, hepatocyte growth factor (HGF) and platelet-derived growth factor (PDGF). In embodiments the anti-cancer agent is decorin itself, and not antibodies to it; and
A soluble and binding but non-functional fragment of any protein involved in an interaction that supports the cancer state, such as a fragment of EGFR or a fragment of EGF, which antagonizes the interaction in which it is normally involved. Proteins of this type serve as decoys that engage the active proteins and inhibit their further contribution to cell division, for instance.

HUCPVCs that express one or more polypeptide antigens derived from and unique to cancer cell surfaces (relative to normal cell surfaces) can be used to deliver anti-cancer vaccines to elicit therapeutic immune responses in the treated cancer subject. Upon administration, genetically modified HUCPVCs express the cancer related antigen that is recognized as foreign by the host immune system. The development of a primary immune response to the antigen, including the activation of the adaptive immune responses (e.g., host antibodies and T cells), allows for the creation of a potent and long-lived secondary response to the presence of the endogenous cancer cells. The polypeptides in vaccines and the identification of immunogenic antigens derived from these are suitable for expression in a HUCPVC. These include protein fusions that comprise the keyhole limpet hemocyanin (KLH) peptide useful to improve immunogenicity in the human host.

Examples of anti-cancer enzymes that can be expressed in HUCPVCs include those pancreatic (proteolytic) enzymes.

HUCPVCs can be genetically modified to express one or more RNA interference (RNAi) molecules when administered to a patient (e.g., a human). RNAi is a mechanism that inhibits gene expression by causing the degradation of specific RNA molecules or hindering the transcription of specific genes. Key to the mechanism of RNAi are small interfering RNA strands (siRNA), which have complementary nucleotide sequences to a targeted messenger RNA (mRNA) molecule. siRNAs are short, single-stranded nucleic acid molecule capable of inhibiting or down-regulating gene expression in a sequence-specific manner; see, for example, Zamore et al., Cell 101:25 33 (2000); Bass, Nature 411:428-429 (2001); Elbashir et al., Nature 411:494-498 (2001); and WO 00/44895; WO 99/32619; WO 00/01846; WO 01/29058; WO 99/07409; and WO 00/44914. Methods of preparing a siRNA molecule for use in gene silencing are described in U.S. Pat. No. 7,078,196, which is hereby incorporated by reference.

The application of RNAi technology (e.g., an siRNA molecule) in the present invention can occur in several ways, each resulting in functional silencing of a gene product in a HUCPVC population. The functional silencing of one or more endogenous HUCPVC gene products may increase the longevity the HUCPVC in vivo (e.g., by silencing one or more pro-apoptotic gene products), or increase the expression of a therapeutic polypeptide.

Functional gene silencing by an RNAi agent (e.g., an siRNA molecule) does not necessarily include complete inhibition of the targeted gene product. In some cases, marginal decreases in gene product expression caused by an RNAi agent can translate to significant functional or phenotypic changes in the host cell, tissue, organ, or animal. Therefore, gene silencing is understood to be a functional equivalent and the degree of gene product degradation to achieve silencing may differ between gene targets or host cell type. Gene silencing may decrease gene product expression by 1%, 2%, 3%, 4%, 5%, 6%, 7%, 8%, 9%, or 10%. Preferentially, gene product expression is decreased by 10%, 20%, 30%, 40%, 50%, 60%, 70%, 80%, 90%, or 100% (i.e., complete inhibition).

In embodiments of the present invention, the polypeptides produced by the HUCPVCs for use in the treatment of cancer are polypeptides that do not have utility as countermeasures, such as those described in WO2009/129616. That is, that published application teaches the use of HUCPVCs that are genetically modified to produce a polypeptide that counteracts challenge by various lethal or debilitating biological or chemical agents, such as those used in warfare and terrorism. Some of the polypeptides listed in that publication are also anti-cancerous in their properties. To the extent there is overlap between what is disclosed in that publication and what is disclosed in this application, the subject matter claimed herein shall have the proviso that any claims to the modified anti-cancer HUCPVCs per se do not include, i.e., exclude, genetic modifications that introduce the ability to produce such overlapping polypeptides. The proviso extends, for instance, to the interferons and interleukins that are disclosed in both documents. The proviso more specifically extends to proteins and oligonucleotides that have utility as countermeasures.

In a related vein, it is noted that genetic modification of HUCPVCs to introduce genes encoding wound healing polypeptides and proteins is described in the Applicant's co-pending patent application having the title: “Genetically Modified Human Umbilical Cord Perivascular Cells For Wound Healing” assigned to Tissue Regeneration Therapeutics Inc. and claiming priority from U.S. Ser. No. 62/350,641 filed Jun. 15, 2016. To the extent any claim in that application or issued patent embraces subject matter in common with the claims of the present application, there is the optional proviso that the overlapping subject matter shall be excluded from the claims of such a patent application. Hence, for the present application, there is optionally the proviso that the HUCPVC populations that are created for use in the present anti-cancer invention exclude those populations in which the HUCPVCs comprise a transgene encoding the same polypeptide or a wound healing polypeptide.

### Genetic Modification of HUCPVCs

Recombinant expression of non-endogenous polypeptides or oligonucleotides in HUCPVCs can be accomplished by using any one of several different standard gene transfer modalities. These modalities, their advantages and constraints, are discussed further below. Exemplary methods of genetically modifying HUCPVCs are also discussed in International Patent Application Publication WO 2007/128115, and in WO2009/129616, both herein incorporated by reference.

Transduction is the infection of a target cell (e.g., a HUCPVC) by a virus that allows genetic modification of the target cell. Many viruses bind and infect mammalian cells and introduce their genetic material into the host cell as part of their replication cycle. Some types of viruses (e.g., retroviruses) integrate their viral genomes into the host's genome. This incorporates the genes of that virus among the genes of the host cell for the life span of that cell. In viruses modified for gene transfer, a donor gene/s (e.g., a humanized monoclonal antibody) is inserted into the viral genome. Additional modifications are made to the virus to improve infectivity or tropism (e.g., pseudotyping), reduce or eliminate replicative competency, and reduce immunogenicity. The newly-introduced mammalian gene will be expressed in the infected host cell or organism and, if replacing a defective host gene, can ameliorate conditions or diseases caused by the defective gene. Adenoviruses and retroviruses (including lentiviruses) are particularly attractive modalities for gene therapy applications, as discussed below, due to the ability to genetically-modify and exploit the life cycle of these viruses.

Recombinant adenoviral vectors offer several significant advantages for the expression of polypeptides (e.g., an antibodies, cytokines, or clotting factors) or oligonucleotides (e.g., an siRNA) in HUCPVCs. The viruses can be prepared at extremely high titer, infect non-replicating cells, and confer high-efficiency and high-level transduction of target cells in vivo after directed injection or perfusion. Furthermore, as adenoviruses do not integrate their DNA into the host genome, this gene therapy modality has a reduced risk of inducing spontaneous proliferative disorders. In animal models, adenoviral gene transfer has generally been found to mediate high-level expression for approximately one week. The duration of transgene expression may be prolonged and ectopic expression reduced, by using tissue-specific promoters. Other improvements in the molecular engineering of the adenoviral vector itself have produced more sustained transgene expression and less inflammation. This is seen with so-called “second generation” vectors harboring specific mutations in additional early adenoviral genes and “gutless” vectors in which virtually all the viral genes are deleted utilizing a cre-lox strategy (Engelhardt et al., Proc. Natl. Acad Sci. USA 91:6196-6200 (1994) and Kochanek et al., Proc. Natl. Acad. Sci. USA 93:5731-5736 (1996)). In addition, recombinant adeno-associated viruses (rAAV), derived from non-pathogenic parvoviruses, can be used to express a polypeptide or oligonucleotide as these vectors evoke almost no cellular immune response, and produce transgene expression lasting months in most systems. Incorporation of a tissue-specific promoter is, again, beneficial.

Other viral vectors useful for the delivery of polypeptides or oligonucleotides into a subject or cells are retroviruses, including lentiviruses. As opposed to adenoviruses, the genetic material in retroviruses is in the form of RNA molecules, while the genetic material of their hosts is in the form of DNA. When a retrovirus infects a host cell, it will introduce its RNA together with some enzymes into the cell. This RNA molecule from the retrovirus will produce a double-stranded DNA copy (provirus) from its RNA molecules through a process called reverse transcription. Following transport into the cell nucleus, the proviral DNA is integrated in a host chromosome, permanently altering the genome of the infected cell and any progeny cells that may arise. The ability to permanently introduce a gene encoding a polypeptide or oligonucleotide into a cell such as a HUCPVC is the defining characteristic of retroviruses used for gene therapy. Retroviruses include lentiviruses, a family of viruses including human immunodeficiency virus (HIV) that includes several accessory proteins to facilitate viral infection and proviral integration.

One problem with using retroviruses for gene therapy is that the integrase enzyme can insert the genetic material of the virus in any arbitrary position in the genome of the host. If genetic material happens to be inserted in the middle of one of the original genes of the host cell, this gene will be disrupted (e.g., insertional mutagenesis). If the gene happens to be one regulating cell division, uncontrolled cell division (e.g., cancer) can occur. This problem has recently begun to be addressed by utilizing zinc finger nucleases or by including certain sequences such as the beta-globin locus control region to direct the site of integration to specific chromosomal sites. Despite this consideration, retroviruses and lentiviruses have considerable utility for gene therapy applications. Current, “third-generation” lentiviral vectors feature total replication incompetence, broad tropism, and increased gene transfer capacity for mammalian cells. Lentiviruses pseudotyped with, e.g., vesicular stomatitis virus glycoprotein (VSV-G) or feline endogenous virus RD114 envelope glycoprotein can be used to transduce HUCPVCs (see, e.g., Zhang et al., “Transduction of bone-marrow-derived mesenchymal stem cells by using lentivirus vectors pseudotyped with modified RD114 envelope glycoproteins,” J. Virol. 78(3):1219-1229 (2004)). U.S. Pat. Nos. 5,919,458, 5,994,136, and 7,198,950, hereby incorporated by reference, describe the production and use of lentiviruses to genetically modify target cells.

Besides adenoviral and retroviral vectors, other viral vectors and techniques are known in the art that can be used to transfer a DNA vector (e.g., a plasmid) encoding a desired polypeptide or oligonucleotide into a subject or cells. These include, e.g., those described by Wattanapitayakul and Bauer (Biomed. Pharmacother 54:487-504 (2000), and citations therein.

Naked DNA or oligonucleotides (e.g., DNA vectors such as plasmids) encoding polypeptides (e.g., an antibody, cytokine, or hormone) or RNA interference molecule (e.g., an siRNA or shRNA) can also be used to genetically modify HUCPVCs. This is the simplest method of non-viral transfection. Clinical trials carried out using intramuscular injection of a naked DNA plasmid have had some success; however expression has been low in comparison to other methods of transfection. Other efficient methods for delivery of naked DNA exist such as electroporation and the use of a “gene gun,” which shoots DNA-coated gold particles into the cell using high pressure gas.

### Cancers

The methods of the invention provide for the administration of genetically modified HUCPVCs to a subject (e.g., humans) presenting with cancer in any of its types and stages.

The term “cancer” as used herein refers to a disease, disorder or condition characterized by cells that have the capacity for autonomous growth or replication, e.g., an abnormal state or condition characterized by proliferative cell growth. The term also refers to a mass of tissue (neoplasm, tumour) resulting from the abnormal growth and/or division of cells in a subject having cancer. Neoplasms can be benign (such as uterine fibroids and melanocytic nevi), potentially malignant (such as carcinoma in situ) or malignant. Exemplary cancer types include but are not limited to carcinoma, sarcoma, metastatic disorders (e.g., tumors arising from the prostate), hematopoietic neoplastic disorders, (e.g., “blood cancers” such as leukemias, lymphomas, myeloma and other malignant plasma cell disorders), metastatic tumors and other cancers. Prevalent cancers include cancers of breast, prostate, colon, lung, liver, brain, ovary and pancreas.

Cancers that can be treated with genetically modified HUCPVCs are numerous and include: Acute Lymphoblastic Leukemia, Acute Myeloid Leukemia, Adrenocortical Carcinoma; AIDS-Related Lymphoma; AIDS-Related Malignancies; Anal Cancer; Astrocytoma, Childhood Cerebellar; Astrocytoma, Childhood Cerebral; Bile Duct Cancer, Extrahepatic; Bladder Cancer; Bone Cancer, Osteosarcoma/Malignant Fibrous Histiocytoma; Brain Stem Glioma; Brain Tumors including Glioma; Cerebellar Astrocytoma; Cerebral Astrocytoma/Malignant Glioma, Ependymoma, Medulloblastoma, and Supratentorial; Primitive Neuroectodermal Tumors; Brain Tumor, Visual Pathway and Hypothalamic Glioma; Breast Cancer; Bronchial Adenomas/Carcinoids; Carcinoid Tumor; Carcinoma, Adrenocortical; Carcinoma, Islet Cell; Carcinoma of Unknown Primary; Central Nervous System Lymphoma, Primary; Cerebellar Astrocytoma; Cerebral Astrocytoma/Malignant Glioma; Cervical Cancer; Chronic Lymphocytic Leukemia; Chronic Myelogenous Leukemia; Chronic Myeloproliferative Disorders; Clear Cell Sarcoma of Tendon Sheaths; Colon Cancer; Colorectal Cancer; Cutaneous T-Cell Lymphoma; Endometrial Cancer; Ependymoma; Epithelial Cancer, Ovarian Cancer; Esophageal Cancer; Ewing's Family of Tumors; Extracranial Germ Cell Tumor; Extragonadal Germ Cell Tumor; Extrahepatic Bile Duct Cancer; Eye Cancer, Intraocular Melanoma; Eye Cancer, Retinoblastoma; Gallbladder Cancer; Gastric (Stomach) Cancer; Gastric (Stomach) Cancer; Gastrointestinal Carcinoid Tumor; Germ Cell Tumor, Extracranial; Germ Cell Tumor, Extragonadal; Germ Cell Tumor, Ovarian; Gestational Trophoblastic Tumor; Glioma; Brain Stem Glioma; Hypothalamic; Hairy Cell Leukemia; Head and Neck Cancer; Hepatocellular (Liver) Cancer, (Primary); Hepatocellular (Liver) Cancer; Hodgkin's Lymphoma; Hypopharyngeal Cancer; Hypothalamic and Visual Pathway Glioma; Intraocular Melanoma; Islet Cell Carcinoma (Endocrine Pancreas); Kaposi's Sarcoma; Kidney Cancer; Laryngeal Cancer; Laryngeal Cancer; Leukemia, Acute Lymphoblastic, Adult; Leukemia, Acute Lymphoblastic, Childhood; Leukemia, Acute Myeloid; Leukemia, Chronic Lymphocytic; Leukemia, Chronic Myelogenous; Leukemia, Hairy Cell; Lip and Oral Cavity Cancer; Liver Cancer; Lung Cancer, Non-Small Cell; Lung Cancer, Small Cell; Lymphoblastic Leukemia; Lymphoblastic Leukemia; Lymphocytic Leukemia, Chronic; Lymphoma, AIDS-Related; Lymphoma, Central Nervous System (Primary); Lymphoma, Cutaneous T-Cell; Lymphoma, Hodgkin's; Lymphoma, Non-Hodgkin's; Lymphoma, Primary Central Nervous System; Macroglobulinemia, Waldenstrom's; Male Breast Cancer; Malignant Mesothelioma; Malignant Thymoma; Medulloblastoma; Melanoma; Melanoma, Intraocular; Merkel Cell Carcinoma; Mesothelioma, Malignant; Metastatic Squamous Neck Cancer with Occult Primary; Multiple Endocrine Neoplasia Syndrome; Multiple Myeloma/Plasma Cell Neoplasm; Mycosis Fungoides; Myelodysplastic Syndromes; Myelogenous Leukemia, Chronic; Myeloid Leukemia, Acute; Myeloma, Multiple; Myeloproliferative Disorders, Chronic; Nasal Cavity and Paranasal Sinus Cancer; Nasopharyngeal Cancer; Nasopharyngeal Cancer; Neuroblastoma; Non-Hodgkin's Lymphoma; Non-Small Cell Lung Cancer; Oral Cancer, Childhood; Oral Cavity and Lip Cancer; Oropharyngeal Cancer; Osteosarcoma/Malignant Fibrous Histiocytoma of Bone; Ovarian Cancer; Ovarian Epithelial Cancer; Ovarian Germ Cell Tumor; Ovarian Low Malignant Potential Tumor; Pancreatic Cancer; Pancreatic Cancer, Islet Cell; Paranasal Sinus and Nasal Cavity Cancer; Parathyroid Cancer; Penile Cancer; Pheochromocytoma; Pineal and Supratentorial Primitive Neuroectodermal Tumors; Pituitary Tumor; Plasma Cell Neoplasm/Multiple Myeloma; Pleuropulmonary Blastoma; Primary Central Nervous System Lymphoma; Primary Liver Cancer, Adult; Primary Liver Cancer, Childhood; Prostate Cancer; Rectal Cancer; Renal Cell (Kidney) Cancer; Renal Cell Cancer; Renal Pelvis and Ureter, Transitional Cell Cancer; Retinoblastoma; Rhabdomyosarcoma, Childhood; Salivary Gland Cancer; Salivary Gland Cancer, Childhood; Sarcoma, Ewing's Family of Tumors; Sarcoma, Kaposi's; Sarcoma (Osteosarcoma)/Malignant Fibrous Histiocytoma of Bone; Sarcoma, Rhabdomyosarcoma Sarcoma, Soft Tissue; Sarcoma, Soft Tissue, Childhood; Sezary Syndrome; Skin Cancer; Skin Cancer (Melanoma); Skin Carcinoma, Merkel Cell; Small Cell Lung Cancer; Small Intestine Cancer; Soft Tissue Sarcoma; Soft Tissue Sarcoma, Childhood; Squamous Neck Cancer with Occult Primary, Metastatic; Stomach (Gastric) Cancer; Stomach (Gastric) Cancer, Childhood; Supratentorial Primitive Neuroectodermal Tumors; T-Cell Lymphoma, Cutaneous; Testicular Cancer; Thymoma; Thymoma, Malignant; Thyroid Cancer; Transitional Cell Cancer of the Renal Pelvis and Ureter; Trophoblastic Tumor, Gestational; Liver and Renal Pelvis, Transitional Cell Cancer; Urethral Cancer; Uterine Sarcoma; Vaginal Cancer; Visual Pathway and Hypothalamic Glioma; Vulvar Cancer; Waldenstrom's Macro globulinemia; and Wilms' Tumor. Metastases of these cancers can also be treated in accordance with the methods described herein.

Subjects that can be Treated with the Genetically Modified HUCPVCs

Subjects that can benefit from the administration of genetically modified HUCPVCs, according to the methods of the invention, to treat, inhibit, reduce, control or prevent cancer and its progression include vertebrates, and especially mammals (e.g., humans, non-human primates (e.g., monkeys, chimpanzees, apes), livestock (e.g., horses, cows, goats, pigs, sheep, deer) and pets including dogs, and cats. In embodiments, the HUCPVCs are similarly extracted from the umbilical cord perivascular tissue of at least the same genus and especially of the same species as the intended recipient.

### Dosing and Administration

The present invention provides genetically modified HUCPVCs that provide a therapeutically effective amount one or more polypeptides (e.g., antibodies, cytokines, or hormones) or oligonucleotides (e.g., siRNAs). Genetically modified HUCPVCs are intended for parenteral (e.g., intramuscular, sub-cutaneous, and intravenous), intranasal, topical, oral, or local administration, such as by a transdermal means, for therapeutic treatment. The genetically modified HUCPVCs are administered parenterally (e.g., by intravenous, intramuscular, or subcutaneous injection) or intraarticular injection at areas affected by the condition. Additional routes of administration include intravascular, intra-arterial, intraperitoneal, intraventricular, epidural, as well as nasal, ophthalmic, intrascleral, intraorbital, rectal, topical, intratumoural or aerosol inhalation administration.

Genetically modified HUCPVCs can be administered for prophylactic or therapeutic treatments. In prophylactic applications, genetically modified HUCPVCs are administered to a subject (e.g., a human) with a clinically determined predisposition or increased susceptibility to cancer progression. For example, HUCPVCs that have been genetically modified to express the her-2 antibody, trastuzumab, can be administered to a subject who presents with her-2+ breast cancer to treat that particular form of cancer. Further, HUCPVC-producing trastuzumab can preferably be co-administered with pertuzumab that is naked or is also produce by the same or a different HUCPVC.

Genetically modified HUCPVCs can be administered to the subject (e.g., a human) in an amount sufficient to delay, reduce, or preferably prevent the progression of clinical disease. In therapeutic applications, genetically modified HUCPVCs are administered to a subject already suffering from cancer to cure or at least partially arrest the cancer symptoms of these agents. The number of HUCPVCs adequate to accomplish this purpose is defined as a “therapeutically effective dose.” Amounts effective for this use may depend on the severity of the disease or condition and the weight and general state of the patient. The total number of genetically modified HUCPVCs administered to a subject in single or multiple doses according to the methods of the invention can be e.g., 10¹, 10², 10³, 10⁴, 10⁵, 10⁶, 10⁷, 10⁸, 10⁹, or more cells, although an effective dose will probably lie in the range of 10³ to 10⁷ cells per dose. Preferably, the genetically modified HUCPVCs are administered to the subject in need thereof in a single dose. Genetically modified HUCPVCs can also be applied as an initial dose followed by booster administrations at one or more hourly, daily, weekly, monthly, or bimonthly intervals. The total effective dose of genetically modified HUCPVCs administered to a subject as a single dose, either as a bolus or by infusion over a relatively short period of time, or can be administered using a fractionated treatment protocol, in which multiple doses are administered over a more prolonged period of time (e.g., a dose every 4-6, 8-12, 14-16, or 18-24 hours, or every 2-4 days, 1-2 weeks, once a month, or once every two months). Alternatively, continuous intravenous infusions sufficient to maintain therapeutically effective concentrations in the blood are contemplated.

The therapeutically-effective amount of a genetically modified HUCPVC to be administered to a subject (e.g., a human) according to the methods of the invention can be determined by a skilled artisan. Factors that can be considered include, e.g., individual differences in the subject's age, weight, condition e.g., the stage and severity at which the cancer is diagnosed and the efficacy of the anti-cancer polypeptide.

The invention provides for the co-administration of a second genetically modified HUCPVC population to a subject (e.g., a human), in which the second HUCPVC population expresses one or more different polypeptides or oligonucleotides for prophylactic or therapeutic applications. Alternatively, one or more mesenchymal stem cells (MSC) that are not HUCPVCs can be co-administered. In this case, the MSC can be genetically modified to express a polypeptide or oligonucleotide. It is not always necessary, however, to administer both HUCPVC and MSC populations at the same time or in the same way. In some cases, the administration of the second population may begin shortly after the completion of the administration period for the first population or vice versa. Such time gap between the two administration periods may vary from one day to one week, to one month, or more. In some cases, two genetically modified HUCPVC populations can be co-administered initially, and subsequently administered singly in following periods (e.g., the administration of two or more HUCPVC populations that individually express a single anti-cancer monoclonal antibody). In addition HUCPVC populations can be modified to express more than one polypeptide or oligonucleotide for prophylactic or therapeutic applications, thus removing the need for multiple administrations. In one embodiment, there is provided a pharmaceutical combination, and the use thereof to treat cancer, comprising a HUCPVC modified genetically to produce trastuzumab and, in combination therewith, a different HUCPVC modified genetically to produce pertuzumab, wherein the combination is administered to treat a cancer subject.

Single or multiple (e.g., 2, 3, 4, 5, 6, 7, 8, 9, 10, 15, 20 or more) administrations of the compositions of the invention that include an effective amount can be carried out with dose levels and pattern being selected by the treating clinician (e.g., a physician or veterinarian). The dose and administration schedule can be determined and adjusted based on the severity and type of cancer agent. Furthermore, a subject (e.g., a mammal, such as a human administered genetically modified HUCPVCs can be monitored throughout the course of treatment according to the methods commonly practiced by clinicians or those described herein.

The invention provides for the co-administration of one or more other anti-cancer agents in combination with genetically modified HUCPVCs. For example, an additional therapeutic agent may be administered with genetically modified HUCPVCs described herein at concentrations known to be effective for such therapeutic agents.

In some instances, the genetically modified HUCPVCs and the additional therapeutic agents (including different HUCPVCs) are administered at least one hour, two hours, four hours, six hours, 10 hours, 12 hours, 18 hours, 24 hours, three days, seven days, fourteen days, or one month or even one year apart. The dosage and frequency of administration of each component can be controlled independently. The additional therapeutic agents described herein may be admixed with additional active or inert ingredients, e.g., in conventional pharmaceutically acceptable carriers. A pharmaceutical carrier can be any compatible, non-toxic substance suitable for the administration of the compositions of the present invention to a subject. Pharmaceutically acceptable carriers include, for example, water, saline, buffers and other compounds, described, for example, in the Merck Index, Merck & Co., Rahway, N.J. A slow release formulation or a slow release apparatus may be also be used for continuous administration. The additional therapeutic regimen may involve other therapies, including modification to the lifestyle of the subject being treated.

In embodiments, therapy entails use of the anti-cancer HUCPVC in combination with a different treatment modality, such as radiation therapy, including external beam radiation. Alternatively, or in addition, a chemotherapeutic agent may be administered to the patient. Preparation and dosing schedules for such chemotherapeutic agents are those suggested in manufacturers' instructions or as determined empirically by the skilled practitioner. The chemotherapeutic agent may precede, or follow administration of the modified HUCPVC or may be given simultaneously therewith.

HUCPVCs that are modified to produce an antibody, trastuzumab for instance, may be combined with chemotherapeutics including irinotecan (CPT-11), cisplatin, cyclophosphamide, melphalan, dacarbazine, doxorubicin, daunorubicin, docetaxel, and topotecan, as well as tyrosine kinase inhibitors, including particularly EGFR kinase inhibitors such as AG1478 ((4-(3-chloroanilino-6,7-dimethoxyquinazoline), gefitinib (Iressa®), erlotinib (Tarceva®), lapatinib (Tykerb®), canertinib (PD183805, Pfizer), PKI-166 (Novartis), PD158780 and pelitinib. It may also be desirable to administer any HUCPVCs that produce HER-2 antibodies in combination with an inhibitor against, such as an antibody against, related tumor associated antigens or their ligands, such as ErbB1 (EGFR, HER-1) ErbB3, ErbB4, or vascular endothelial factor (VEGF), and/or antibodies that bind to EGF or TGFα or PDGF.

In another embodiment of the invention, there is provided an article of manufacture containing the genetically modified HUCPVCs in a population useful for the treatment of the cancers described herein. The article of manufacture comprises a container and a label. Suitable containers include, for example, bottles, vials, syringes, and test tubes. The containers may be formed from a variety of materials such as glass or plastic. The container holds a composition which is effective for treating the condition and may have a sterile access port (for example the container may be an intravenous solution bag or vial having a stopper pierceable by a hypodermic injection needle). The label on or associated with the container indicates that the composition is used for treating a cancer condition. The article of manufacture may further compromise a second container compromising a pharmaceutically-acceptable buffer, such as phosphate-buffered saline, Ringer's solution and dextrose solution. It may further include other matters desirable from a commercial end use standpoint, including other buffers, diluents, filters, needles, syringes, and package inserts with instructions for use.

In embodiments, the HUCPVCs are provided in a frozen state such as a cryogenic state useful to store the cells over time. The cells can then be thawed and formulated for subsequent use to treat a cancer patient.

In specific embodiments of the present invention, the HUCPVC is modified genetically to produce trastuzumab and is administered to treat a subject presenting with breast cancer, especially HER2+ breast cancer. In another embodiment, the HUCPVC is modified genetically to produce pertuzumab and is administered to treat a subject presenting with breast cancer, especially HER2+ breast cancer. In a specific embodiment, the subject with breast cancer is treated with a single HUCPVC species modified genetically to produce both pertuzumab and trastuzumab, or is treated with different HUCPVC species, each one being modified genetically to produce one or the other of pertuzumab and trastuzumab.

In specific embodiments of the present invention, the HUCPVC is modified genetically to produce an EGFR antibody such as cetuximab or panitumumab and is administered to treat a subject presenting with head and neck cancer, or colorectal cancer.

In another specific embodiment, the HUCPVC is modified genetically to produce an immune checkpoint inhibitor, including particularly an antibody that is selected from ipilimumab, atezolizumab, pembrolizumab and novolumab for the treatment of prostate cancer. In combination, ipilimumab and novolumab are used particularly to treat melanoma.

In another specific embodiment, the HUCPVC is modified genetically to produce an immune modulator, including particularly the antibody rituximab or ofatumumab, for the treatment of a subject presenting with lymphomas and leukemias, including non-Hodgkin's lymphoma and lymphocyte predominant subtype of Hodgkin's lymphoma.

In a further specific embodiment, the HUCPVC is modified genetically to produce TRAIL, for the treatment of a subject presenting with blood cancers including leukemias as well as solid tumours, of prostate for instance.

In an aspect of the present invention, the various HUCPVC populations are assembled, to provide an inventory of ready-to-use drugs. The assemblage comprises separately packaged HUCPVC populations, each differing in their genetic modification. For instance, one HUCPVC population could be genetically modified to produce an anti-cancer antibody, and another HUCPVC population could be genetically modified to produce either a drug useful in combination with that antibody, or any other polypeptide that is useful in the treatment of cancer. In an embodiment, the assemblage comprises one population of HUCPVCs that produces trastuzumab and another separately stored HUCPVC population that produces pertuzumab. In another embodiment, the different populations produce ipilimumab and novolumab. In yet another embodiment, one population produces a first protein and another population produces a second protein that binds with the first protein to produce an anti-cancer polypeptide. The assemblage will have an organized compilation of containers (vials, tubes, wells, etc.) containing each distinct population in a properly formulated, e.g., freeze-dried form for storage. The inventory will further be catalogued in a database that identifies and/or locates each population and its characteristics relevant to cancer therapy.

### EXAMPLES

The following examples are to illustrate the invention. They are not meant to limit the invention in any way.

### Example 1: Production of HER-2 Antibody, Trastuzumab

As used herein, the terms “HER-2” and “erbB2” are used interchangeably with reference to any protein that comprises the expressed and processed product of the HER-2 gene, wherein the protein is designated as UniProtKB/Swiss-Prot P04626-1. This is the receptor for such ligands as EGF. The HER-2 antibody known as trastuzumab comprises both a heavy chain and a light chain, having the primary sequences shown below:

Entire Light chain
[SEQ ID No. 1]
DIQMTQSPSSLSASVGDRVTITCRASQDVNTAVAWYQQKPGKAPKLLIY
SASFLYSGVPSRFSGSRSGTDFTLTISSLQPEDFATYYCQQHYTTPPTF
GQGTKVEIKRTVAAPSVFIFPPSDEQLKSGTASVVCLLNNEYPREAKVQ
WKVDNALQSGNSQESVTEQDSKDSTYSLSSTLTLSKADYEKHKVYACEV
THQGLSSPVTKSFNRGEC;
Entire Heavy chain
[SEQ ID No. 2]
EVQLVESGGGLVQPGGSLRLSCAASGFNIKDTYIHWVRQAPGKGLEWVA
RIYPTNGYTRYADSVKGRFTISADTSKNTAYLQMNSLRAEDTAVYYCSR
WGGDGFYAMDYWGQGTLVTVSSASTKGPSVFPLAPSSKSTSGGTAALGC
LVKDYFPEPVTVSWNSGALTSGVHTFPAVLQSSGLYSLSSVVTVPSSSL
GTQTYICNVNHKPSNTKVDKKVEPKSCDTPPPCPRCPAPELLGGPSVFL
FPPKPKDTLMISRTPEVTCVVVDVSHEDPEVKFNWYVDGVEVHNAKTKP
REEQYNSTYRVVSVLTVLHQDWLNGKEYKCKVSNKALPAPIEKTISKAK
GQPREPQVYTLPPSRDELTKNQVSLTCLVKGFYPSDIAVEWESNGQPEN
NYKTTPPVLDSDGSFFLYSKLTVDKSRWQQGNVFSCSVMHEALHNHYTQ
KSLSLSPGK

### Genetic Payload: Trastuzumab

A plasmid containing DNA encoding the genes for both light and heavy chains of trastuzumab was purchased from Addgene.org (https://www.addgene.org/61883). To generate the adenovirus both genes were cloned into a shuttle vector that was later used to produce the recombinant adenoviral plasmid by homologous recombination in E. coli. The gene sequence can be found here: https://www.addgene.org/61883/sequences/#depositor-full

### In Vitro Evaluation

Transduced HUCPVCs are tested for trastuzumab expression using standard analysis, such as ELISA. For purposes of identifying the HER-2 protein on disease cells that can be targeted by HUCPVC-produced trastuzumab (and structurally similar antibodies) the commercial test known as HerceptTest® can conveniently be used. This is a semi-quantitative immunohistochemical assay for determination of HER-2 protein overexpression in breast cancer tissues. Positive or negative results aid in the classification of abnormal cells/tissues and provide a basis for treatment with HER-2 antibody.

Using Western blot, known amounts of supernatant can be separated on a SDS-PAGE as described and then transferred onto a Hybond-N nitrocellulose membrane for 1 h at 275 mA. The membrane is blocked for 1 hour in 0.15% Tween 20, 5% skimmed milk in PBS and incubated for 1 hour with an anti-human IgG conjugated to Cy5 (Jackson, Cat#109-176-099). The trastuzumab signal is revealed by scanning with the Typhoon Trio+.

For analysis by ELISA, 96 well/plates are coated with 50 μl of AffiniPure Goat Anti-Human IgG, (H+L) (Jackson Immuno Research) and incubated overnight at 4° C. The wells are washed with PBS and incubated for 30 minutes at 37° C. with 100 μl of 1% BSA in PBS at 37° C. Then, 25 μl of samples diluted with 1% BSA in PBS are added to the wells, which are incubated for 2 hrs at 37° C. The wells are washed with 0.05% Tween 20 in PBS and incubated with an alkaline Phosphatase-conjugated AffiniPure Goat Anti-Human IgG (H+L) for 1 hour at 37° C. The wells are washed with 0.05% Tween 20 in PBS, followed by PBS. The trastuzumab signal is then revealed by incubation with PNPP for 30 min at 37° C. The signal intensity can be measured at 405 nm. A standard curve can then be made using known amount of purified antibody (IgG1, kappa from myeloma plasma).

To purify the antibody, the supernatant is concentrated with a Amicon Ultra (Ultracel-50K) at 1500 rpm to a volume of 500 μl. The antibody is purified using the Nab spin kit Protein A mini column (Thermo Scientific) according to the manufacture's recommendations. The purified antibodies are then desalted and resuspended in PBS using the desalting column PD-10 (GE Healthcare). The antibodies then are concentrated by centrifugation on an Amica Ultra 100,000 MWCO membrane. The purified trastuzumab is quantified by reading the optical density at 280 nm using a Nanodrop spectrophotometer. The purified antibody can be kept frozen at −20° C. in 50% glycerol.

Binding of the antibody to erbB2 on the cancer cell surface can be determined using flow cytometry. For this purpose, cells are plated such that they were not more than 80% confluent on the day of analysis. Tumor cells overexpressing HER-2 (SkBr3, ˜2.5M Her2/cell or BT474, ˜3M Her2/cell) or normal (human cardiac myocytes, ˜20,000 Her2/cell) are washed in PBS and harvested by the addition of cell dissociation buffer (Sigma.). A cell suspension containing 2.5×10⁵ in 500 μl corresponding cell culture media) is incubated with various concentrations (0.01-100 ug/ml) of anti-HER2 antibodies for 2 hours at 4° C. (to prevent internalization). Following 1 wash with cell culture media, primary antibody is incubated with 2 ug Dylight 488 conjugated AffiniPure goat anti-human IgG Alexa 488 secondary antibody (Jackson ImmunoResearch 109-487-003) in 100 ul of media for 1 h at 4° C. Cells are then pelleted and stored on ice until ready to analyzed by flow cytometry. Prior to analysis, cell pellets are resuspended in 300-500 ul media and filtered through a 50 um nylon mesh filter to remove cell aggregates. Flow cytometry analyses are performed on 10,000 viable cells gated on forward scattering, side scattering parameters and propidium iodide dye exclusion using a BD LSRII flow Cytometer (Becton-Dickinson Biosciences, CA, USA) and a standard filter set using BD FACSDiva™ acquisition software.

A commercial source of trastuzumab (Roche) can be used as a benchmark for comparison purposes.

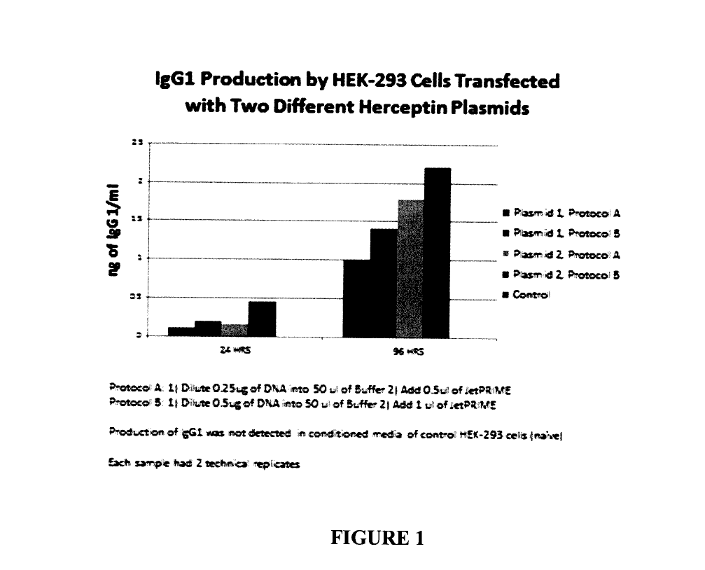

The production of trastuzumab IgG1 from different HEK293 populations is revealed in FIG. 1. As shown, expression increases with time. It is anticipated that HUCPVC cells will exhibit similar properties.

### Example 2: HUCPVCs Produce Interferons

By “interferon” is meant an anti-cancer mammalian (e.g., a human) interferon-alpha, -beta, -gamma, or -tau polypeptide, or biologically-active fragment thereof, e.g., IFN-α (e.g., IFN-α-1a; see e.g., U.S. Patent Application No. 2007/0274950, incorporated by reference herein), IFN-α-1b, IFN-α-2a (see PCT Application No. WO 07/044083, incorporated by reference herein), and IFN-α-2b), IFN-β (e.g., described in U.S. Pat. No. 7,238,344, incorporated by reference herein; IFN-b-1a (AVONEX® and REBIF®), as described in U.S. Pat. No. 6,962,978, incorporated by reference herein, and IFN-β-1b (BETASERON®, as described in U.S. Pat. Nos. 4,588,585; 4,959,314; 4,737,462; and 4,450,103; incorporated by reference herein), IFN-g, and IFN-t (as described in U.S. Pat. No. 5,738,845 and U.S. Patent Application Publication Nos. 2004/0247565 and 2007/0243163; incorporated by reference herein).

The recombinant expression of interferons in HUCPVCs that are administered to a cancer patient can provide broad spectrum activity. Interferons (IFNs) produced by genetically modified HUCPVCs can be used to treat patients presenting with different cancers. When administered per se for the treatment of cancer, IFNs exhibit a short in vivo half-life. Administration of HUCPVCs genetically modified to express one or more IFNs (e.g., IFN-alpha) overcomes this shortcoming by providing extended release and delivery of the IFN. Standard clinical administration of IFNs requires frequent injections or modification (e.g., pegylation) due to the rapid decay kinetics of IFN. In the present example, this problem is overcome by providing IFN-alpha using a genetically modified HUCPVC.

### Genetic Payload: Interferon Alpha (IFN-α)

HUCPVCs are transduced with a retroviral vector (e.g., a lentivirus) that encodes interferon-alpha. Upon integration of the proviral DNA into the HUCPVC chromosome, a constitutively active promoter (e.g., a CMV promoter) is used to drive expression and secretion of the IFN-α.

### In Vitro Evaluation of IFN-α Production

Transduced HUCPVCs are tested for IFN-α expression using standard immunobloting analysis, such as ELISA, Western blot, dot blot, or immunoprecipitation IFN-α activity is confirmed by antiproliferative assays as described by Foser et al., “Improved biological and transcriptional activity of monopegylated interferon-alpha-2a isomers,” The Pharmacogenomics Jour. 3:312-319 (2003).

### Example 3: HUCPVCs Express the Anti-Cancer Protein, TRAIL

A premade adenovirus expressing TRAIL (tumor necrosis factor superfamily member 10 (TNFSF10), transcript variant 1 having the nucleotide sequence at reference NM_003810, was obtained at (http://www.vigenebio.com/ORF/human/VH866841/TNFSF10-adeno). The protein sequence of TRAIL is:

[SEQ ID No. 3]
MAMMEVQGGPSLGQTCVLIVIFTVLLQSLCVAVTYVYFTNELKQMQDKY
SKSGIACFLKEDDSYWDPNDEESMNSPCWQVKWQLRQLVRKMILRTSEE
TISTVQEKQQNISPLVRERGPQRVAAHITGTRGRSNTLSSPNSKNEKAL
GRKINSWESSRSGHSFLSNLHLRNGELVIHEKGFYYIYSQTYFRFQEEI
KENTKNDKQMVQYIYKYTSYPDPILLMKSARNSCWSKDAEYGLYSIYQG
GIFELKENDRIFVSVTNEHLIDMDHEASFFGAFLVG;

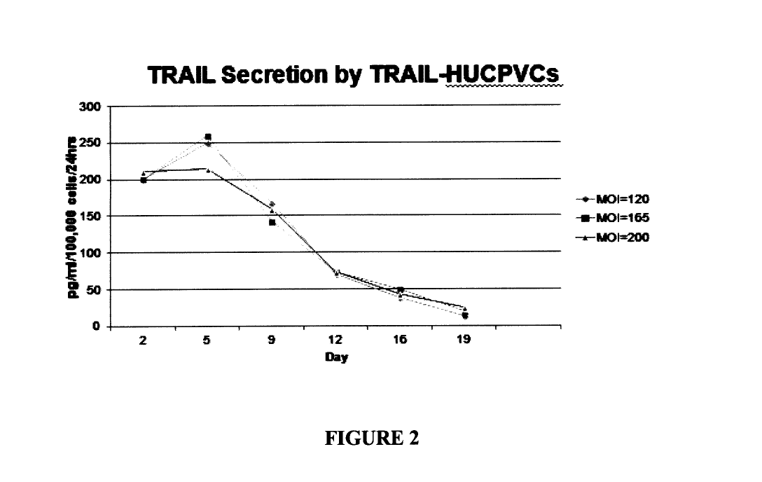

HUCPVCs were seeded at a cell density of 29,000 cells/cm² in a total volume of 142 μL/cm² of medium. After cell adhesion, HUCPVCs were transduced with Ad-TRAIL at various multiplicities of infection. TRAIL-HUCPVCs conditioned media were collected at different time points. Samples were tested for expression of TRAIL using ELISA. Results are presented in FIG. 2.

### Example 4: HUCPVCs Produce Interleukin-10

A premade adenovirus expressing IL-10 (interleukin 10—nucleotide sequence at reference NM_000572), was obtained at http://www.vigenebio.com/ORF/human/VH869610/IL10-adeno). The protein sequence of IL-10 is:

[SEQ ID No. 4]
MHSSALLCCLVLLTGVRASPGQGTQSENSCTHFPGNLPNMLRDLRDAFS
RVKTFFQMKDQLDNLLLKESLLEDFKGYLGCQALSEMIQFYLEEVMPQA
ENQDPDIKAHVNSLGENLKTLRLRLRRCHRFLPCENKSKAVEQVKNAFN
KLQEKGIYKAMSEFDIFINYIEAYMTMKIRN;

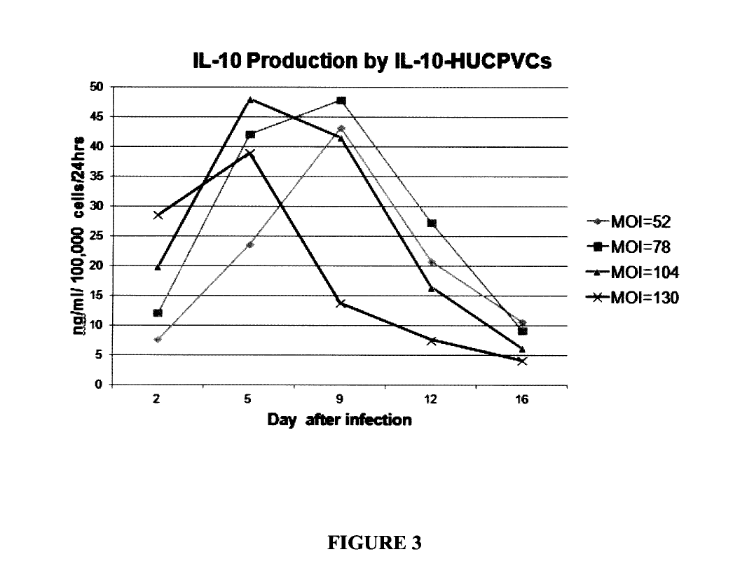

HUCPVCs were seeded at a cell density of 29000 cells/cm² in a total volume of 142 μL/cm² of medium. After cell adhesion, HUCPVCs were transduced with Ad-IL-10 at various multiplicities of infection. IL-10-HUCPVCs conditioned media were collected at different time points. Samples were tested for IL-10 expression using ELISA. Results are provided in FIG. 3.

### Example 5: Genetically Modified HUCPVCs Expressing Decorin

To determine how much decorin (Dcn) protein is secreted by native HUCPVCs, and whether they can be engineered to secrete higher than endogenous Dcn levels, 100,000 HUCPVCs (Lot 130, P5) were seeded into each well of a 6 well plate. Two constructs were used for genetic engineering: a recombinant adenovirus (pAd5) encoding the full human decorin gene (pAd5-Dcn) and pAd5-CAR-Dcn, which encodes human decorin fused to the CAR peptide that homes to the vasculature and thus can target the fusion protein to wounds.

Both pAd5 constructs used include an internal ribosome entry site (IRES) upstream of an eGFP transgene; this reporter construct produces an eGFP molecule each time a Den molecule is produced, and is useful for validating transfection efficiency and transgene expression. The eGFP is not fused to the Dcn protein, but is simply an expression level reporter. Twenty four hours after seeding, cells were incubated for 2 hours with a minimal volume of either media alone (for native cells), or media containing the pA5-Dcn construct at an MOI (multiplicity of infection, the ratio of infective particles to the number of cells) of 20 or 100. These MOIs were selected to initially assess the range in which cells should be engineered to maximize exogenous Dcn expression without toxic effects to the cells. After 2 hours, the virus cocktail was removed and the media replaced. Conditioned media (CM) was collected from the cultured cells and replaced every 72 hours, and stored at −20° C. until analysis.

The amount of Dcn present in CM from the native and engineered HUCPVC cells was quantified by enzyme-linked immunosorbent assay (ELISA) (AbCam human ELISA kit, ab99998). Samples were analyzed in duplicate as neat, or diluted to 1/10, 1/100 and 1/1000. Only 1/100 or 1/1000 dilutions were within the linear range of the assay, depending on the sample. A standard curve was plotted, and the amount of Dcn present in each sample extrapolated using absorbance readings within the linear range. The limit of detection for the assay was set at 1.2, or 20% above the absorbance of the lowest standard.

Genetically modified HUCPVCs secreted Dcn and CAR-Dcn into the culture medium. Dcn was detected in CM from native HUCPVCs, and at significantly higher levels in HUCPVCs genetically modified to express Dcn or CAR-Dcn. Further, HUCPVCs secrete more decorin as a consequence of higher transgene copy number.

Twenty four hours after engineering, eGFP was observed in approximately 20% of cells engineered at MOI 100. eGFP accumulated in these cells, as evidenced by increased frequency and intensity of eGFP, and nearly all cells were eGFP positive by day 3. eGFP was extremely faint and barely discernible in cells engineered at MOI 20. Cells engineered at MOI 100 began to exhibit morphological signs of toxicity by day 3 after engineering. By day 7, cells began to detach and dead cells were evident in the culture media. The study was terminated at day 9, as the MOI 100 cultures were too compromised for reliable data analysis.

The amount of Dcn and CAR-Den secreted by HUCPVCs was greater on day 6 as compared to day 3 post-engineering. On day 9, many MOI 100 cells had already begun to detach from the culture vessel and dead cells were evident in the media. Den levels present in CM at day 9, though were comparable to Den levels in CM observed at day 6. The presence of fewer viable cells at day 9 may be a consequence of eGFP accumulation in these cells or may be related to the very high levels of Dcn secreted by cells engineered with many copies of the Den transgene at MOI 100.

The samples analyzed here are 72 hour media collections. The half-life of Dcn has been reported as 2.5 hours in cell culture although its metabolism by HUCPVCs in particular is unknown. Hence, these data may only represent a snapshot of the amount of Den in the CM. The quantity of Dcn produced by the cells over a 24 hour period, for example, may in fact be much higher. In addition, the eGFP is produced from the same promoter in the current pAd5 constructs; it is expected that Dcn expression will further increase when den is expressed under a dedicated promoter.

Conditioned medium (CM) samples quantified by ELISA were also analyzed by Western blot. Proteins from conditioned media samples analyzed by ELISA (see above) were diluted 1:10, separated by denaturing sodium dodecyl sulfate (SDS) gel electrophoresis, transferred to a polyvinylidene fluoride (PVDF) membrane, and probed using an anti-human decorin antibody. Consistent with the ELISA data, the band intensity from MOI 100 was higher than for MOI 20, and these blots validate the presence of the Dcn protein in CM from native and engineered HUCPVCs, as well as the presence of CAR-Den from engineered HUCPVCs. In each of the experimental samples, the Den band appears as a sharp band, not a smear. According to literature, smeared bands on a Den Western blot are typical for recombinant protein samples, and represent heterogeneity in chondroitin sulfate chains. These observations were validated in a duplicate experiment using an anti-Dcn antibody raised against a different epitope.

The genetically modified HUCPVCs can be kept frozen (around −70/80° C.) or in a cryogenic state (liquid nitrogen/−196° C.) following their transfection. Cold storage of the cells can be achieved by the method described in greater detail in the Applicant's WO2007/071048, I incorporated herein by reference. The present invention thus includes in some embodiments the genetically modified anti-cancer HUCPVCs in a frozen state suitable for storage over time. The cells exhibit expression of the anti-cancer transgene rapidly when thawed, and can be formulated and used directly for cancer treatment. The present invention thus includes the process in which the genetically modified HUCPVCs are obtained in a frozen state, and are then thawed and then formulated to provide a composition useful to treat a cancer subject.

### Other Embodiments

While the invention has been described in connection with specific embodiments thereof, it will be understood that it is capable of further modifications and this application is intended to cover any variations, uses, or adaptations of the invention following, in general, the principles of the invention and including such departures from the present disclosure that come within known or customary practice within the art to which the invention pertains and may be applied to the essential features hereinbefore set forth.

All publications and patent applications mentioned in this specification are herein incorporated by reference to the same extent as if each independent publication or patent application was specifically and individually indicated to be incorporated by reference in their entirety.

1. A method for treating cancer in a subject, comprising administering to the subject a human umbilical cord perivascular cell (HUCPVC) that has been genetically modified to increase the expression of an oligonucleotide or a polypeptide in said HUCPVC relative to a HUCPVC that has not been genetically modified, wherein said modified HUCPVC treats said cancer.

2. The method of claim 1, wherein the HUCPVC is genetically modified to express a transgene that encodes an anti-cancer polypeptide.

3. The method according to claim 2, wherein the anti-cancer polypeptide is an antibody or an anti-cancer fragment thereof.

4. The method according to claim 3, wherein the anti-cancer polypeptide is an antibody or a fragment thereof that is an immune checkpoint inhibitor.

5. The method according to claim 3, wherein the antibody or fragment thereof binds and inhibits an immune checkpoint selected from CTLA-4, PD-1, PD-1 receptor, PD-L1, PD-L2, CD20, CD25, CD27L, CD28, CD30L, CD40, CD40L, CD47, CD200, SIRPa, toll-like receptors TLR3, TLR7 and TLR8, VCP, PLIF, LSF-1, Nip, uromodulin, Fas, FasL, SIRPα, B7.1, and B7.2.

6. The method according to claim 5, wherein the antibody or fragment thereof binds an immune checkpoint selected from CTLA-4, PD-1, PD-1 receptor and PD-1 receptor ligands designated PD-L1 and PD-L2.

7. The method according to claim 4, wherein the immune checkpoint inhibitor is an antibody selected from ipilimumab (CTLA-4), atezolizumab (PD-1), pembrolizumab or nivolumab (PD-1 receptor), rituximab (CD20), and ofatumumab (CD20).

8. The method according to claim 5, wherein the anti-cancer polypeptide is an antibody or fragment thereof that binds to a growth factor or a growth factor receptor.

9. The method according to claim 8, wherein the antibody or fragment thereof binds to a growth factor selected from transforming growth factor-beta (TGF-β) superfamily, platelet derived growth factor (PGDF), insulin-like growth factors (IGFs), epidermal growth factor (EGF), vascular endothelial growth factor (VEGF), hepatocyte growth factor (HGF), keratinocyte growth factor (KGF), glucagon-like peptide 2 (GLP-2).

10. The method according to claim 9, wherein the antibody is bevacizumab.

11. The method according to claim 8, wherein the antibody or fragment thereof binds to a receptor for a growth factor selected from transforming growth factor-beta (TGF-β) superfamily, platelet derived growth factor (PGDF), insulin-like growth factors (IGFs), epidermal growth factor (EGF), vascular endothelial growth factor (VEGF), hepatocyte growth factor (HGF), keratinocyte growth factor (KGF), glucagon-like peptide 2 (GLP-2)

12. The method according to claim 11, wherein the antibody or fragment thereof binds to the her-2 gene expression product.

13. The method according to claim 11, wherein the HUCPVC has been genetically modified to produce the antibody trastuzumab.

14. The method according to claim 11, wherein the HUCPVC has been genetically modified to produce the antibody pertuzumab.

15. The method according to claim 11, wherein the antibody or fragment thereof binds to EGFR.

16. The method according to claim 15, wherein the HUCPVC has been genetically modified to produce an EGFR antibody selected from cetuximab, panitumumab, matuzumab, necitumumab, nimotuzumab, and zalutumab.

17. The method according to claim 2, wherein the anti-cancer polypeptide is an interleukin.

18. The method according to claim 17, wherein the anti-cancer polypeptide is interleukin-2.

19. The method according to claim 2, wherein the anti-cancer polypeptide is an interferon.

20. The method according to claim 2, wherein the anti-cancer polypeptide is TRAIL.

21. The method according to claim 2, wherein the anti-cancer polypeptide is a member of the tumour necrosis factor superfamily.

22. The method according to claim 2, wherein the anti-cancer polypeptide is a decorin.

23. The method according to claim 2, wherein the anti-cancer polypeptide is an antibody that binds a GD2 ganglioside.

24. The method according to claim 2, wherein the anti-cancer polypeptide is an antibody that binds to and inhibits a baculoviral IAP repeat-containing protein selected from BIRC1, BIRC2, BIRC3, BIRC4, BIRC5 (survivin), BIRC6, BIRC7 and BIRC8.

25. The method according to claim 2, wherein the anti-cancer polypeptide is an antibody that is basiliximab or daclizumab.

26. The method according to claim 2, wherein the anticancer polypeptide is an Fc fusion protein comprising an anti-cancer polypeptide.

27. The method according to claim 2, wherein the anti-cancer polypeptide is a fragment of a polypeptide involved in an interaction that supports cancer progression, the fragment being an antagonist of that interaction.

28. The method according to claim 2, wherein the anti-cancer polypeptide stimulates growth of a cell type useful in cancer therapy selected from granulocyte macrophage colony-stimulating factor (GM-CSF), granulocyte colony-stimulating factor (G-CSF), macrophage colony-stimulating factor (M-CSF), erythropoietin (EPO), and thrombopoietin (TPO).

29. The method of claim 1, wherein said oligonucleotide is selected from an RNA interference (RNAi) molecule capable of inhibiting oncogene expression.

30. The method of claim 29, wherein said RNAi molecule is a small inhibitory RNA (siRNA) or short hairpin RNA (shRNA) molecule.

31. The method of claim 1, wherein said method comprises administering a single dose of a composition comprising said HUCPVC to said subject.

32. The method of claim 1, wherein said HUCPVC is administered as a vaccine to protect said subject.

33. The method of claim 1, wherein said polypeptide is a cellular protein that acts as an antigen, thereby generating an immune response in said subject against said antigen.

34. The method of claim 1, wherein said cell persists in said subject for greater than one week.

35. The method of claim 34, wherein said HUCPVC persists in said subject for greater than one month.

36. The method of claim 35, wherein said HUCPVC persists in said subject for greater than two months.

37. The method of claim 35, wherein said HUCPVC persists in said subject for greater than six months.

38. The method of claim 1, wherein said HUCPVC is administered to said subject intravenously, intramuscularly, orally, by inhalation, parenterally, intraperitoneally, intraarterially, transdermally, sublingually, nasally, transbuccally, liposomally, adiposally, opthalmically, intraocularly, subcutaneously, intrathecally, topically, or locally.

39. The method of claim 1, wherein said HUCPVC evades immune recognition in said subject.

40. The method of claim 1, wherein said subject is administered between 10¹ and 10¹³ HUCPVCs per dose.

41. The method of claim 40, wherein said subject is administered between 10³ and 10⁸ HUCPVCs per dose.

42. The method of claim 1, wherein said HUCPVC has been genetically modified to express two or more oligonucleotides or polypeptides.

43. The method of claim 1 further comprising administering at least one mesenchymal stem cell (MSC), wherein said MSC is not a HUCPVC.

44. The method of claim 43, wherein said MSC has been genetically modified to increase the expression of an oligonucleotide or a polypeptide in said MSC relative to a MSC that has not been genetically modified.

45. The method of claim 44, wherein said MSC is isolated from bone marrow, umbilical cord blood, adipose tissue, embryonic yolk sac, placenta, skin, or blood.

46. The method of claim 1, wherein said HUCPVC is administered with a pharmaceutically acceptable carrier or excipient.

47. The method of claim 1, wherein said oligonucleotide or polypeptide is endogenous to said HUCPVC.

48. The method of claim 1, wherein said oligonucleotide or polypeptide is not endogenous to said HUCPVC.

49. The method of claim 1 comprising administering at least two different HUCPVC populations, wherein each HUCPVC population has been genetically modified to increase the expression of an anti-cancer polypeptide in each HUCPVC population, wherein the anti-cancer polypeptide is different in each HUCPVC populations.

50. The method of claim 1, wherein the HUCPVC is genetically modified to produce two different polypeptides that cooperate to provide an anti-cancer benefit to the subject.

51. The method according to claim 50, wherein the two different polypeptides are an antibody light chain and an antibody heavy chain.

52. The method of claim 1, wherein said HUCPVC is allogeneic to said subject.

53. A kit, or an article of manufacture, comprising a human umbilical cord perivascular cell (HUCPVC) that has been genetically modified to increase the expression of an oligonucleotide or a polypeptide in said HUCPVC relative to a HUCPVC that has not been genetically modified, wherein said HUCPVC controls cancer in a recipient.

54. A human umbilical cord perivascular cell that has been genetically modified for use in a method according to any one of claims 1-52.

55. A pharmaceutical composition comprising a pharmaceutically acceptable carrier and genetically modified human umbilical cord perivascular cells according to claim 54, wherein said cells are present in a dose effective for use in accordance with a method according to any one of claims 1-52.

56. An assemblage comprising individual and separate containers each comprising a HUCPVC population according to claim 54, and a catalog indexing the containers in the assemblage, wherein each HUCPVC is modified genetically to produce a different polypeptide useful in the treatment of cancer.

57. The method according to any one of claims 1-52, wherein the cancer is selected from Acute Lymphoblastic Leukemia, Acute Myeloid Leukemia, Adrenocortical Carcinoma; AIDS-Related Lymphoma; AIDS-Related Malignancies; Anal Cancer; Astrocytoma, Childhood Cerebellar; Astrocytoma, Childhood Cerebral; Bile Duct Cancer, Extrahepatic; Bladder Cancer; Bone Cancer, Osteosarcoma/Malignant Fibrous Histiocytoma; Brain Stem Glioma; Brain Tumors including Glioma; Cerebellar Astrocytoma; Cerebral Astrocytoma/Malignant Glioma, Ependymoma, Medulloblastoma, and Supratentorial; Primitive Neuroectodermal Tumors; Brain Tumor, Visual Pathway and Hypothalamic Glioma; Breast Cancer; Bronchial Adenomas/Carcinoids; Carcinoid Tumor; Carcinoma, Adrenocortical; Carcinoma, Islet Cell; Carcinoma of Unknown Primary; Central Nervous System Lymphoma, Primary; Cerebellar Astrocytoma; Cerebral Astrocytoma/Malignant Glioma; Cervical Cancer; Chronic Lymphocytic Leukemia; Chronic Myelogenous Leukemia; Chronic Myeloproliferative Disorders; Clear Cell Sarcoma of Tendon Sheaths; Colon Cancer; Colorectal Cancer; Cutaneous T-Cell Lymphoma; Endometrial Cancer; Ependymoma; Epithelial Cancer, Ovarian Cancer; Esophageal Cancer; Ewing's Family of Tumors; Extracranial Germ Cell Tumor; Extragonadal Germ Cell Tumor; Extrahepatic Bile Duct Cancer; Eye Cancer, Intraocular Melanoma; Eye Cancer, Retinoblastoma; Gallbladder Cancer; Gastric (Stomach) Cancer; Gastric (Stomach) Cancer; Gastrointestinal Carcinoid Tumor; Germ Cell Tumor, Extracranial; Germ Cell Tumor, Extragonadal; Germ Cell Tumor, Ovarian; Gestational Trophoblastic Tumor; Glioma; Brain Stem Glioma; Hypothalamic; Hairy Cell Leukemia; Head and Neck Cancer; Hepatocellular (Liver) Cancer, (Primary); Hepatocellular (Liver) Cancer; Hodgkin's Lymphoma; Hypopharyngeal Cancer; Hypothalamic and Visual Pathway Glioma; Intraocular Melanoma; Islet Cell Carcinoma (Endocrine Pancreas); Kaposi's Sarcoma; Kidney Cancer; Laryngeal Cancer; Laryngeal Cancer; Leukemia, Acute Lymphoblastic, Adult; Leukemia, Acute Lymphoblastic, Childhood; Leukemia, Acute Myeloid; Leukemia, Chronic Lymphocytic; Leukemia, Chronic Myelogenous; Leukemia, Hairy Cell; Lip and Oral Cavity Cancer; Liver Cancer; Lung Cancer, Non-Small Cell; Lung Cancer, Small Cell; Lymphoblastic Leukemia; Lymphoblastic Leukemia; Lymphocytic Leukemia, Chronic; Lymphoma, AIDS-Related; Lymphoma, Central Nervous System (Primary); Lymphoma, Cutaneous T-Cell; Lymphoma, Hodgkin's; Lymphoma, Non-Hodgkin's; Lymphoma, Primary Central Nervous System; Macroglobulinemia, Waldenstrom's; Male Breast Cancer; Malignant Mesothelioma; Malignant Thymoma; Medulloblastoma; Melanoma; Melanoma, Intraocular; Merkel Cell Carcinoma; Mesothelioma, Malignant; Metastatic Squamous Neck Cancer with Occult Primary; Multiple Endocrine Neoplasia Syndrome; Multiple Myeloma/Plasma Cell Neoplasm; Mycosis Fungoides; Myelodysplastic Syndromes; Myelogenous Leukemia, Chronic; Myeloid Leukemia, Acute; Myeloma, Multiple; Myeloproliferative Disorders, Chronic; Nasal Cavity and Paranasal Sinus Cancer; Nasopharyngeal Cancer; Nasopharyngeal Cancer; Neuroblastoma; Non-Hodgkin's Lymphoma; Non-Small Cell Lung Cancer; Oral Cancer, Childhood; Oral Cavity and Lip Cancer; Oropharyngeal Cancer; Osteosarcoma/Malignant Fibrous Histiocytoma of Bone; Ovarian Cancer; Ovarian Epithelial Cancer; Ovarian Germ Cell Tumor; Ovarian Low Malignant Potential Tumor; Pancreatic Cancer; Pancreatic Cancer, Islet Cell; Paranasal Sinus and Nasal Cavity Cancer; Parathyroid Cancer; Penile Cancer; Pheochromocytoma; Pineal and Supratentorial Primitive Neuroectodermal Tumors; Pituitary Tumor; Plasma Cell Neoplasm/Multiple Myeloma; Pleuropulmonary Blastoma; Primary Central Nervous System Lymphoma; Primary Liver Cancer, Adult; Primary Liver Cancer, Childhood; Prostate Cancer; Rectal Cancer; Renal Cell (Kidney) Cancer; Renal Cell Cancer; Renal Pelvis and Ureter, Transitional Cell Cancer; Retinoblastoma; Rhabdomyosarcoma, Childhood; Salivary Gland Cancer; Salivary Gland Cancer, Childhood; Sarcoma, Ewing's Family of Tumors; Sarcoma, Kaposi's; Sarcoma (Osteosarcoma)/Malignant Fibrous Histiocytoma of Bone; Sarcoma, Rhabdomyosarcoma Sarcoma, Soft Tissue; Sarcoma, Soft Tissue, Childhood; Sezary Syndrome; Skin Cancer; Skin Cancer (Melanoma); Skin Carcinoma, Merkel Cell; Small Cell Lung Cancer; Small Intestine Cancer; Soft Tissue Sarcoma; Soft Tissue Sarcoma, Childhood; Squamous Neck Cancer with Occult Primary, Metastatic; Stomach (Gastric) Cancer; Stomach (Gastric) Cancer, Childhood; Supratentorial Primitive Neuroectodermal Tumors; T-Cell Lymphoma, Cutaneous; Testicular Cancer; Thymoma; Thymoma, Malignant; Thyroid Cancer; Transitional Cell Cancer of the Renal Pelvis and Ureter; Trophoblastic Tumor, Gestational; Liver and Renal Pelvis, Transitional Cell Cancer; Urethral Cancer; Uterine Sarcoma; Vaginal Cancer; Visual Pathway and Hypothalamic Glioma; Vulvar Cancer; Waldenstrom's Macro globulinemia; and Wilms' Tumor.

58. Use of a human umbilical cord perivascular cell according to claim 54 for the treatment of cancer.

59. In a frozen state, a human umbilical cord perivascular cell that has been genetically modified for use in a method according to any one of claims 1-52.
